(19) United States
(12) Patent Application Publication
(10) Pub. No.: US 2023/0301530 A1
(43) Pub. Date: Sep. 28, 2023

(54) APPARATUS AND METHODS FOR OPTIMIZING BLOOD PRESSURE MEASUREMENTS

(71) Applicant: GE Precision Healthcare LLC, Wauwatosa, WI (US)

(72) Inventor: Panu Takala, Helsinki (FI)

(21) Appl. No.: 17/655,946

(22) Filed: Mar. 22, 2022

(51) Int. Cl. A61B 5/0225; A61B 5/022; A61B 5/00

(52) U.S. Cl. CPC A61B 5/0225; A61B 5/02233; A61B 5/721

(57) ABSTRACT

An apparatus comprising an inflatable member, a sensor and a controller. The inflatable member is configured to apply pressure to a portion of a body of a patient and is configured to be placed in a deflated configuration, a first inflated configuration, and a second inflated configuration. A pressure within the inflatable member when the inflatable member is in the second inflated configuration is greater than a pressure within the inflatable member when the inflatable member is in the first inflated configuration. The sensor is configured to detect vibration. The controller is operatively coupled to the sensor and to the inflatable member. The controller is configured to prevent the inflatable member from being placed in the second inflated configuration in response to the sensor detecting vibrations indicating movement of the body of the patient while the inflatable member is in the first inflated configuration.

### TECHNICAL FIELD

This disclosure relates generally to blood pressure measurement devices, and more specifically, to blood pressure measurement devices that are configured to optimize blood pressure measurements.

### BACKGROUND

Non-invasive blood pressure measuring devices are frequently used to measure the blood pressure of an individual. For example, blood pressure measurements of a patient in a hospital or doctor's office are frequently taken. In some cases, inflatable blood pressure cuffs may be used to measure the blood pressure of a patient. A variety of circumstances may cause the measurement of the blood pressure of a patient to be inaccurate. For example, measurements of the blood pressure of a patient taken by a blood pressure cuff may be inaccurate if the patient is moving too much during the taking of the blood pressure measurement. Additionally, measurements of the blood pressure of a patient taken while the patient is startled or surprised may be inaccurate. Accordingly, there is a need for apparatus and methods for taking accurate blood pressure measurements.

### SUMMARY

In a general aspect, an apparatus includes an inflatable member, a sensor and a controller. The inflatable member is configured to apply pressure to a portion of a body of a patient and is configured to be placed in a deflated configuration, a first inflated configuration, and a second inflated configuration. A pressure within the inflatable member when the inflatable member is in the second inflated configuration is greater than a pressure within the inflatable member when the inflatable member is in the first inflated configuration. The sensor is configured to detect vibration. The controller is operatively coupled to the sensor and to the inflatable member. The controller is configured to prevent the inflatable member from being placed in the second inflated configuration in response to the sensor detecting vibrations indicating movement of the body of the patient while the inflatable member is in the first inflated configuration.

In another aspect, an apparatus includes a controller, an output coupled to the controller, and an input coupled to the controller. The output is configured to provide a signal to inflate an inflatable member. The inflatable member is configured to apply pressure to a portion of a body of a patient and is configured to be placed in a deflated configuration, a first inflated configuration, and a second inflated configuration. A pressure within the inflatable member when the inflatable member is in the second inflated configuration is greater than a pressure within the inflatable member when the inflatable member is in the first inflated configuration. The controller is configured to prevent the inflatable member from being placed in the second inflated configuration in response to the input receiving a signal indicating movement of the body of the patient while the inflatable member is in the first inflated configuration.

In another aspect, a method, includes inflating an inflatable member from a deflated configuration to a first inflated configuration to apply pressure to a portion of a body of a patient; detecting movement of the body of the patient; and preventing the inflatable member from inflating from the first inflated configuration to a second inflated configuration in response to detecting movement of the body of the patient.

### BRIEF DESCRIPTION OF THE DRAWINGS

FIG. 1 is a block diagram of an apparatus according to an aspect.

FIG. 2 is a perspective view of an apparatus according to an aspect.

FIG. 3 is a perspective view of the apparatus of FIG. 2 disposed on an arm of a patient.

FIG. 4 is a cross-sectional view of an inflation member of the apparatus of FIG. 2 disposed on an arm of a patient in a deflated configuration.

FIG. 5 is a cross-sectional view of the inflation member of FIG. 2 disposed on an arm of a patient in a partially inflated configuration.

FIG. 6 is a cross-sectional view of the inflation member of FIG. 2 disposed on an arm of a patient in an inflated configuration.

FIG. 7 is a block diagram of a control unit of the apparatus of FIG. 2.

FIGS. 8-11 are flow charts of a methods according to aspects.

### DETAILED DESCRIPTION

In general, the implementations are directed to medical devices and methods. The term patient or user may hereinafter be used for a person who benefits from the medical devices or the methods disclosed in the present disclosure. For example, the patient can be a person whose body contacted by, engaged with, or otherwise interacts with the medical device.

In some implementations, an apparatus or device is configured to take blood pressure measurements of a patient. In some cases, the apparatus or device is configured to take blood pressure measurements of a patient that have a likelihood of being accurate. For example, in some implementations, the apparatus or device is configured to take blood pressure measurements while a patient is not moving or is not moving their hand or arm. Accordingly, in some implementations, artifacts or other vibrations may not be detected during the blood pressure measurement of the patient.

Additionally, in some implementations, the apparatus or device provides notice to the patient that a blood pressure measurement will be taken. In such implementations, the patient may refrain from moving, such as moving their hand or their arm, during the blood pressure measurement. Furthermore, such notice to the patient may allow the patient to mentally prepare for the blood pressure measurement. Accordingly, the blood pressure measurement may not surprise or startle the patient. In some implementations, the techniques disclosed herein are able provide accurate blood pressure measurements.

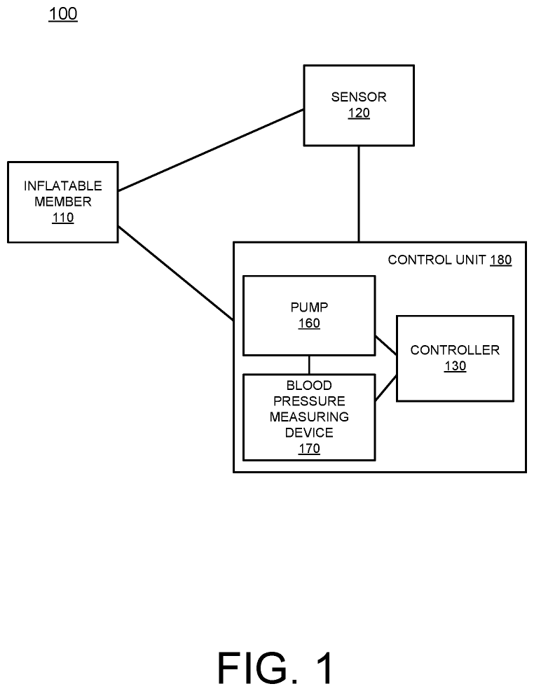

FIG. 1 is a block diagram of an apparatus 100 according to an aspect. The apparatus 100 includes an inflatable member 110, a sensor 120, and a controller 130. In some embodiments, the inflatable member 110 is configured to be placed in a variety of different states or inflation configurations. For example, in some embodiments, the inflatable member 110 is configured to be placed in a deflated configuration, a first inflated (or partially inflated) configuration, and a second inflated (or fully or more fully inflated) configuration. The pressure within the inflatable member 110 is greater when the inflatable member 110 is disposed in the second inflated configuration than when the inflatable member 110 is disposed in the first inflated configuration. Similarly, the pressure within the inflatable member 110 is greater when the inflatable member 110 is in the first inflated configuration than when the inflatable member 110 is in the deflated configuration. In some embodiments, the inflatable member 110 is configured to be disposed proximate a portion of a body of a patient and apply varying amounts of pressure against the body of the patient (depending on how inflated the inflatable member is). For example, the inflatable member 110 may place more pressure on a portion of the body of the patient when the inflatable member 110 is disposed in the second inflated configuration than when the inflatable member 110 is disposed in the first inflated configuration. In some embodiments, the inflatable member 110 includes a chamber, bladder, or other cavity that is configured to receive air or other fluid to be placed in a deflated state, a partially inflated state, or a fully (or more fully) inflated state.

In some embodiments, the inflatable member 110 is a cuff, such as a blood pressure cuff. In such embodiments, the inflatable member 110 is configured to be removably coupled to a portion of a body of a patient. For example, in some embodiments, the inflatable member 110 is configured to be removably coupled to a portion of a body of a patient, such as a portion of an arm of the patient or a portion of a leg of the patient.

The sensor 120 is configured to detect vibrations. For example, in some embodiments, the sensor 120 is configured to detect vibrations associated with the flow of blood within a body of a patient. In some embodiments, the sensor 120 is operatively coupled to the inflatable member 110 such that when the inflatable member 110 is removably coupled to a portion of the body of the patient, the sensor 120 is positioned to detect vibrations within the body of the patient, such as vibrations associated with the flow of blood within the body of the patient. In some embodiments, the sensor 120 is also configured to detect vibrations associated with movement the body of the patient. For example, in some embodiments, when the inflatable member 110 is coupled to an arm of a patient, the sensor 120 is positioned and configured to detect vibrations associated with movement of the body of the patient. In some cases, the sensor 120 is configured to detect movement of the arm, hand, fingers, or other portion of the body of the patient.

In some embodiments, the sensor 120 is a pressure transducer or other transducer that is configured to detect vibrations or movement. In other embodiments, the sensor 120 is a different type of sensor configured to detect vibrations or movement.

The controller 130 is operatively coupled to the inflatable member 110 and to the sensor 120. The controller 130 is configured to prevent the inflatable member from being placed in a fully or more fully inflated configuration in response to the sensor detecting vibrations indicating movement of the body of the patient while the inflatable member is in the partially inflated configuration.

For example, in some embodiments, an output is operatively coupled to the controller and an input is operatively coupled to the controller 130. The output is configured to provide a signal to inflate an inflatable member. The input is configured to receive a signal when motion is detected by the sensor 120. The controller 130 is configured to prevent the inflatable member 110 from being placed in the fully or more fully inflated configuration in response to the input receiving the signal indicating movement of the body of the patient. In some embodiments, the controller 130 is configured to prevent the inflatable member 110 from being placed in the fully or more fully inflated configuration in response to the input receiving the signal indicating movement of the body of the patient while the inflatable member 110 is in the partially inflated configuration.

In some embodiments, the controller 130 includes hardware and software. For example, various implementations of the controller 130 described herein can be realized as described in detail below.

In the illustrated embodiment, the controller 130 is operatively coupled to or is a portion of a control unit 180. In the illustrated embodiment, the control unit 180 is operatively coupled to the inflatable member 110 and to the sensor 120. The control unit 180 includes a pump or a pump system 160 and a blood pressure measuring device 170.

The pump or pump system 160 is operatively coupled to the inflatable member 110. The pump or pump system 160 is configured to selectively inflate and deflate the inflatable member 110. For example, in some embodiments, the inflatable member 110 includes an inflation portion or a bladder that is configured to receive a fluid such as air. The pump or pump system 160 is configured to deliver the fluid or air to the inflatable portion of the inflatable member 110 to selectively place the inflatable member 110 in different states of inflation (a deflated state, a partially inflated state, and a fully or more fully inflated state).

In some embodiments, the pump or pump system 160 includes a pump or a series of pumps and a valve or a series of valves. The pump system 160 may be configured to inflate the inflatable member 110 and to selectively deflate the inflatable member 110. For example, in some embodiments, the pump or pump system 160 is configured to inflate the inflatable member 110 to a pressure greater than a systolic blood pressure of a patient and is configured to allow the inflatable member 110 to slowly deflate. In some embodiments, the slow deflation of the inflatable member 110 allows the blood pressure of the patient to be measured. In some embodiments, the inflatable member 110 may be deflated continuously. In other embodiments, the inflatable member 110 may be deflated in a step-wise manner.

The pump or pump system 160 is disposed within the control unit 180 in the illustrated embodiment. In other embodiments, the pump or pump system 160 is house or disposed at a different location. For example, the pump or pump system 160 may be housed or disposed within a unit separate from the control unit 180. In some embodiments, the pump or the pump system 160 may be housed or located within the inflatable member 110.

The blood pressure measuring device 170 is operatively coupled to the inflatable member 110 and to the sensor 120. The blood pressure measuring device 170 is configured to receive inputs or information from the sensor 120 and is configured to determine or calculate a blood pressure of a patient. For example, in some embodiments, the blood pressure measuring device 170 is configured to determine or calculate a blood pressure of a patient as the inflatable member 110 is allowed to deflate from a fully or more fully inflated configuration.

In some embodiments, the blood pressure measuring device 170 includes a computer program or module that is configured to receive data inputs and to provide a blood pressure measurement of the patient as an output. The blood pressure measuring device 170 is disposed within the control unit 180 in the illustrated embodiment. In other embodiments, the blood pressure measuring device 170 may be housed or located within a unit separate from the control unit 180.

In use, the apparatus 100 may be placed such that at least a portion of the apparatus 100 is disposed proximate a portion of a body of a patient. For example, the apparatus 100 may be placed such that the inflatable member 110 is disposed proximate a portion of the body of the patient so that the inflatable member 110 is able to place or apply pressure on the portion of the body of the patient. In some embodiments, the inflatable member 110 is coupled to or disposed around a limb of the patient, such as an arm or a leg of the patient. In such embodiments, the inflatable member 110 may be configured to surround a portion of the limb of the patient. In some embodiments, the inflatable member 110 is coupled to or disposed around the limb of the patient while the inflatable member 110 is in a deflated state.

The inflatable member 110 can then be inflated to a partially inflated state. For example, in some embodiments, the inflatable member 110 may be inflated to a partially inflated state such that the pressure within the inflatable member 110 is slightly lower than the diastolic blood pressure of the patient or to a pressure slightly lower than a typical diastolic blood pressure of a patient population. In some embodiments, the pump or pump system 160 may function to inflate the inflatable member 110 to the partially inflated state.

In some embodiments, the inflation of the inflatable member 110 to a partially inflated state may provide a tactile notification, an auditory notification, or both to the patient. Accordingly, the patient may be notified that that a blood pressure measurement is about to be taken. Upon receiving the notification, the patient may conscientiously refrain from moving their body so that an accurate blood pressure reading may be taken. Additionally, upon receiving the notification, the patient may be more relaxed during the blood pressure measurement as the sudden inflation of the inflatable member 110 will not come as a surprise to the patient. In such cases, the lack of patient surprise may prevent an inaccurate blood pressure measurement.

While the inflatable member 110 is in the partially inflated state, the sensor 120 may detect vibrations. For example, vibrations caused by movement of the patient, such as movement of the hand or arm of the patient, may be detected by the sensor 120. As the sensor 120 is used to detect blood flow within the body of the patient, additional vibrations detected by the sensor 120 that are not related to blood flow within the patient may cause a blood pressure measurement to be inaccurate. For example, detection of artifacts (such as vibrations caused by movement of the patient) by the sensor 120 may cause the blood pressure measurement to be inaccurate.

If the sensor 120 detects vibrations while the inflatable member 110 is in the partially inflated state, the controller 130 is configured to prevent the inflatable member 110 from being inflated to its fully or more fully inflated state. Accordingly, a measurement of the blood pressure of the patient is delayed. For example, in some embodiments, the measurement of the blood pressure of the patient may be delayed until a time when the sensor 120 does not detect vibrations associated with movement of the patient.

If the sensor 120 does not detect vibrations while the inflatable member 110 is in the partially inflated state, the controller 130 is configured to allow the inflatable member 110 to inflate to its fully or more fully inflated state. In such cases, the pump or pump system 160 may inflate the inflatable member 110 to the more fully inflated state and may allow the inflatable member 110 to slowly deflate. While the inflatable member 110 is deflating, the blood pressure measuring device 170 may then receive signals from the sensor 120 related to the blood flow within the body of the patient and determine the blood pressure of the patient.

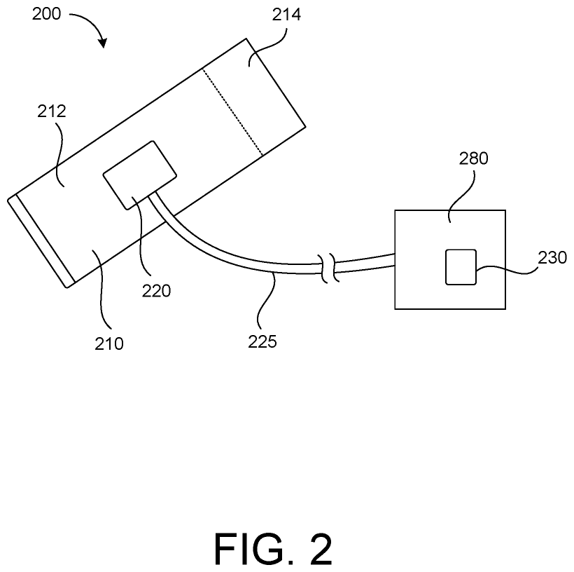

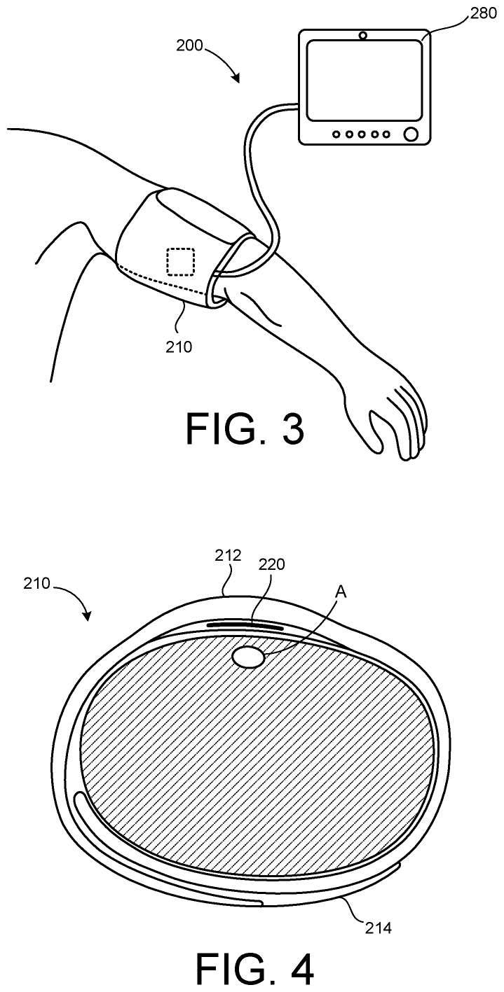

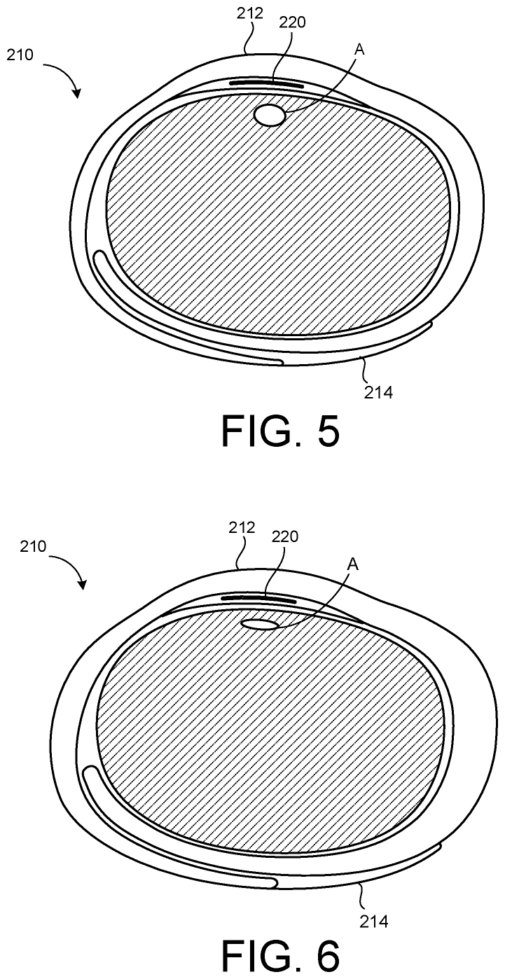

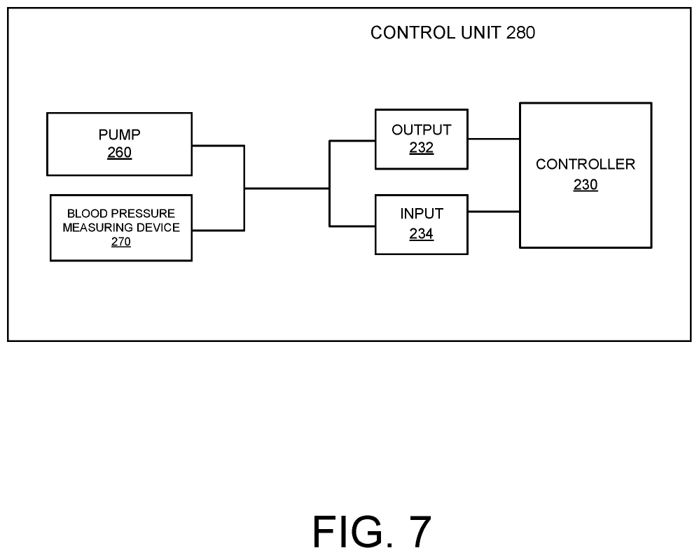

FIGS. 2-7 illustrate an apparatus 200 according to an aspect. FIG. 2 is a perspective view of the apparatus 200 according to an aspect. FIG. 3 is a perspective view of the apparatus 200 disposed on an arm of a patient. FIG. 4 is a cross-sectional view of an inflatable member 210 of the apparatus 200 disposed on an arm of a patient in a deflated configuration. FIG. 5 is a cross-sectional view of the inflatable member 210 disposed on an arm of a patient in a partially inflated configuration. FIG. 6 is a cross-sectional view of the inflatable member 210 disposed on an arm of a patient in an inflated configuration. FIG. 7 is a block diagram of a control unit 280 of the apparatus 200.

As best illustrated in FIG. 2, the apparatus 200 includes an inflatable member 210, a sensor 220, and a controller 230 disposed within the control unit 280. The inflatable member 210 includes an inflatable portion 212 and a coupling portion 214. In some embodiments, the inflatable portion 212 of the inflatable member 210 is configured to receive a fluid such as air to place the inflatable member 210 in a variety of inflation states. For example, in some embodiments, the inflatable portion 212 may include a cavity, chamber, bladder, or the like that is configured to receive different amounts of fluid or air to place the inflatable member 210 in a variety of different inflation states.

In the illustrated embodiment, the inflatable member 210 is configured to be placed in a deflated configuration (as best illustrated in FIG. 4), a first inflated (or partially inflated) configuration (as best illustrated in FIG. 5), and a second inflated (or fully or more fully inflated) configuration (as best illustrated in FIG. 6). The pressure within the inflatable portion 212 of the inflatable member 210 is greater when the inflatable member 210 is disposed in the second inflated configuration than when the inflatable member 210 is disposed in the first inflated configuration.

In some embodiments, the inflatable member 210 has an internal pressure less than the diastolic blood pressure of a patient when the inflatable member 210 is disposed in the partially inflated configuration (as best illustrated in FIG. 5). In some embodiments, the inflatable member 210 has an internal pressure greater than the systolic blood pressure of a patient when the inflatable member is in the inflated configuration (as best illustrated in FIG. 6).

The inflatable member 210 is configured to be disposed proximate a portion of a body of a patient and apply varying amounts of pressure against the body of the patient (depending on how inflated the inflatable member is). For example, the inflatable member 210 places more pressure on a portion of the body of the patient when the inflatable member 210 is disposed in the second inflated configuration than when the inflatable member 210 is disposed in the first inflated configuration.

The inflatable member 210 is a cuff, such as a blood pressure cuff. As best illustrated in FIG. 3 the inflatable member 210 is configured to be removably coupled to a portion of a body of a patient. Specifically, the inflatable member 210 is configured to be removably coupled to an arm of a patient such that the inflatable member 210 surrounds a portion of the arm of the patient.

In the illustrated embodiment, the coupling portion 214 of the inflatable member 210 may be used to removably couple the inflatable member 210 to an arm of a user. Specifically, the inflatable member 210 may be wrapped around an arm of the patient and the coupling portion 214 may be removably coupled to another portion of the inflatable member 210 to form a loop or cuff around the arm of the patient.

In some embodiments, the coupling portion 214 includes hook material or loop material or a different type of material that is configured to be coupled to, such as removably coupled to, a different portion of the inflatable member 210.

The sensor 220 is configured to detect vibrations. Specifically, the sensor 220 is configured to detect vibrations associated with the flow of blood within a body of a patient. In the illustrated embodiment, the sensor 220 is operatively coupled to the inflatable member 210. As best illustrated in FIGS. 4-6, the sensor 220 is coupled to the inflatable member 210 such that when the inflatable member 210 is removably coupled to an arm of the patient, the sensor 220 is positioned to detect vibrations within the body of the patient, such as vibrations associated with the flow of blood within the body of the patient. For example, the sensor 220 may be disposed such that the sensor 220 is configured to detect vibrations associated with blood flow within artery A of the patient.

In the illustrated embodiment, the sensor 220 is also configured to detect vibrations associated with movement the body of the patient. When the inflatable member 210 is coupled to the arm of the patient, the sensor 220 is positioned and configured to detect vibrations associated with movement of the body of the patient. In some cases, the sensor 220 is configured to detect movement of the arm, hand, fingers, or other portion of the body of the patient.

In some embodiments, the sensor 220 is a pressure or other transducer that is configured to detect vibrations or movement. In other embodiments, the sensor 220 is a different type of sensor configured to detect vibrations or movement.

The control unit 280 and the controller 230 are operatively coupled to the inflatable member 210 and to the sensor 220. The control unit 280 also includes a pump or pump system 260 and a blood pressure measuring device 270. In the illustrated embodiment, the control unit 280 and thus the controller 230, the pump or pump system 260, and the blood pressure measuring device 270 are operatively coupled to the inflatable member 210 and to the sensor via a conduit 225. While the illustrated embodiment includes the pump or pump system 260, the blood pressure measuring device 270, and the controller 230 disposed within a single control unit 280, in other embodiments, such components may be housed or disposed in different units. Additionally, in some embodiments, the control unit 280 may be wirelessly coupled to the inflatable member 210. For example, in some embodiments, the control unit 280 may be operatively coupled to the inflatable member 210 via infrared wave, radio frequency, Bluetooth or other wireless technology.

The pump or pump system 260 is operatively coupled to the inflatable member 210. The pump or pump system 260 is configured to selectively inflate and deflate the inflatable member 210. In some embodiments, the pump or pump system 260 includes a pump or series of pumps and a valve or a series of valves.

In some embodiments, the inflatable member 210 includes an inflatable portion 212 that is configured to receive a fluid such as air. In some embodiments, the inflatable portion 212 includes a bladder, cavity or other chamber that is configured to receive fluid such as air. The pump or pump system 260 is configured to deliver the fluid or air to the inflatable portion 212 of the inflatable member 210 to selectively place the inflatable member 210 in different states of inflation (a deflated state, a partially inflated state, and a fully or a more fully inflated state).

In some embodiments, the pump or pump system 260 is configured to allow the inflatable member 210 to slowly deflate after it is placed in the fully or more fully inflated configuration. For example, a valve or a valve system of the pump system 260 may allow the inflatable member 210 to slowly deflate.

The blood pressure measuring device 270 is operatively coupled to the inflatable member 210 and to the sensor 220. The blood pressure measuring device 270 is configured to receive inputs or information from the sensor 220 and is configured to determine or calculate a blood pressure of a patient.

The controller 230 is configured to prevent the inflatable member 210 from being placed in the second inflated configuration or a more fully inflated configuration in response to the sensor detecting vibrations indicating movement of the body of the patient while the inflatable member 210 is in the first or partially inflated configuration.

As best illustrated schematically in FIG. 7, an output 232 is operatively coupled to the controller and an input 234 is operatively coupled to the controller 230. The output 232 is configured to provide a signal to inflate the inflatable member 210. For example, in the illustrated embodiment, the output 232 is configured to provide a signal to the pump or pump system 260 to inflate the inflatable member 210. In other embodiments, the output 232 is configured to provide a signal to a different component.

The input 234 is configured to receive a signal when motion is detected by the sensor 220. For example, the input 234 is configured to receive a signal from the sensor 220 when the sensor detect bodily motion such as movement of the hand or arm of the patient. The controller 230 is configured to prevent the inflatable member 210 from being placed in the fully or more fully inflated configuration in response to the input 234 receiving the signal indicating movement of the body of the patient. For example, in some embodiments, the controller 230 may refrain from sending a signal to the pump or pump system 260 instructing the pump or pump system 260 to inflate the inflatable member 210. In other embodiments, the controller 230 may send a signal to the pump or pump system 260 instructing the pump or pump system 260 to not inflate or not further inflate the inflatable member 210.

In some embodiments, the controller 230 is configured to prevent the inflatable member 210 from being placed in the fully or more fully inflated configuration in response to the input 234 receiving the signal indicating movement of the body of the patient while the inflatable member 210 is in the partially inflated configuration.

In some embodiments, the controller 230 includes hardware and software. For example, various implementations of the controller 230 described herein can be realized as described in detail below.

In use, the apparatus 200 may be placed such that at least a portion of the apparatus 200 is disposed proximate a portion of a body of a patient. For example, the apparatus 200 may be placed such that the inflatable member 210 is disposed proximate a portion of the body of the patient so that the inflatable member 210 is able to place pressure on the portion of the body of the patient. In some embodiments, the inflatable member 210 is coupled to or disposed around a limb of the patient, such as an arm or a leg of the patient. In some embodiments, the inflatable member 210 is configured to be coupled to the body of the patient such that it surrounds the arm of the patient. In some embodiments, the inflatable member 210 is coupled to or disposed around the limb of the patient while the inflatable member 210 is in a deflated state.

The inflatable member 210 can then be inflated to a partially inflated state. For example, in some embodiments, the inflatable member 210 may be inflated to a partially inflated state such that the pressure within the inflatable member 210 is slightly lower than the diastolic blood pressure of the patient or to a pressure slightly lower than a typical diastolic blood pressure of a patient population. In some embodiments, the pump or pump system 260 may function to inflate the inflatable member 210 to the partially inflated state based on a signal received from the controller 230.

In some embodiments, the inflation of the inflatable member 210 to a partially inflated state may provide a tactile notification, an auditory notification, or both to the patient. Accordingly, the patient may be notified that that a blood pressure measurement is about to be taken. Upon receiving the notification, the patient may conscientiously refrain from moving their body so that an accurate blood pressure reading may be taken. Additionally, upon receiving the notification, the patient may be more relaxed during the blood pressure measurement as the sudden inflation of the inflatable member 210 will not come as a surprise (and thereby may prevent an inaccurate blood pressure measurement).

While the inflatable member 210 is in the partially inflated state, the sensor 220 may detect vibrations. For example, such vibrations may be caused by movement of the patient, such as movement of the hand or arm of the patient. As the sensor 220 is used to detect blood flow within the body of the patient, additional vibrations detected by the sensor 220 that are not related to blood flow within the patient may cause a blood pressure measurement to be inaccurate.

If the sensor 220 detects vibrations while the inflatable member 210 is in the partially inflated state, the controller 230 is configured to prevent the inflatable member 210 from being inflated to its fully or more fully inflated state. Accordingly, a measurement of the blood pressure of the patient is delayed. For example, in some embodiments, the measurement of the blood pressure of the patient may be delayed until a time when the sensor 220 does not detect vibrations associated with movement of the patient.

If the sensor 220 does not detect vibrations while the inflatable member 210 is in the partially inflated state, the controller 230 is configured to allow the inflatable member 210 to inflate to its fully or more fully inflated state. For example, the controller 230 may be configured to send a signal to cause the inflatable member 210 to inflate to its more fully inflated state. For example, the controller 230 may be configured to send a signal to the pump or pump system 260. In such cases, the pump or pump system 260 may inflate the inflatable member 210 to the more fully inflated state and may allow the inflatable member 210 to slowly deflate. The blood pressure measuring device 270 may then receive signals from the sensor 220 related to the blood flow within the body of the patient and determine the blood pressure of the patient.

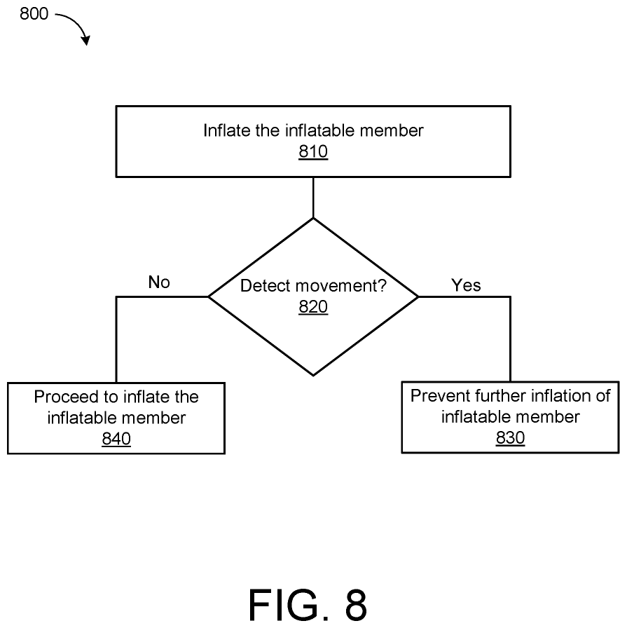

FIG. 8 is a flow chart of a method 800 according to an aspect. At 810, the inflatable member (such as inflatable member 210) is disposed proximate a portion of a body of a patient and the inflatable member is inflated. In some embodiments, the inflatable member is partially inflated. For example, the inflatable member may be inflated to a pressure below the diastolic blood pressure of the patient.

At 820, it is determined whether there is patient movement. For example, in some embodiments, a sensor (such as sensor 220) is used to detect patient movement. For example, the sensor may detect if there is any patient movement that might cause or potentially contribute to an inaccurate blood pressure measurement. In some embodiments, the sensor may detect movement in a hand, arm, fingers, or other body part of the patient. If at 820 the sensor detects movement, a signal may be sent to a controller (such as controller 230). At 830, the inflatable member is prevented from further inflating. For example, the inflatable member may be prevented from inflating to the fully or more fully inflated state. In some embodiments, the controller is configured to receive the signal from the sensor indicating that the sensor has detected movement of the patient. The controller may be configured to send a signal to a pump or a pump system to prevent the further inflating of the inflatable member.

If the sensor does not detect patient movement, then at 840, the inflatable member may be allowed to be placed in the fully or more fully inflated state. For example, in some embodiments, the sensor may send a signal to the controller indicating that patient movement was not detected and the controller may cause the pump or pump system to place the inflatable member in the more fully inflated state. In some embodiments, a blood pressure measurement of the patient may be taken once the inflatable member is placed in the fully or more fully inflated state.

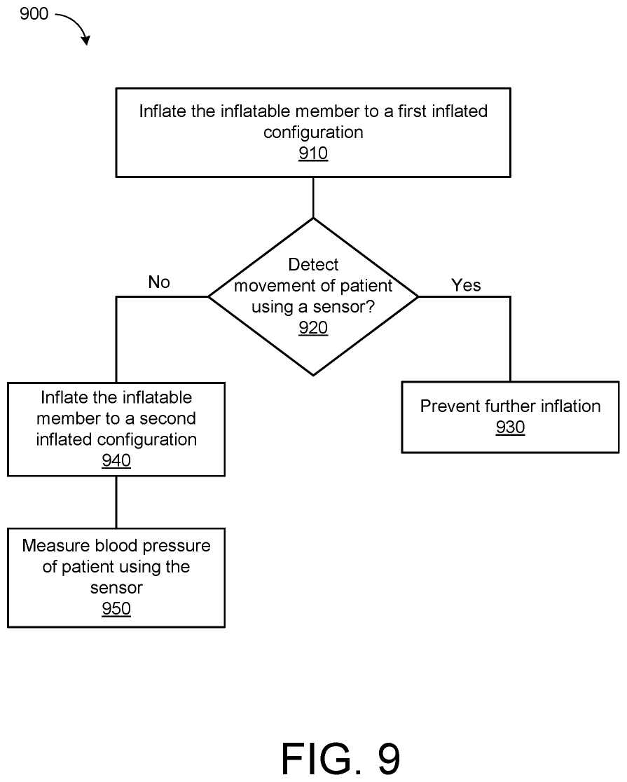

FIG. 9 is a flow chart of a method 900 according to an aspect. At 910, the inflatable member (such as inflatable member 210) is disposed proximate a portion of a body of a patient and the inflatable member is inflated to a first inflated configuration. In some embodiments, the inflatable member 210 is inflated to a first inflated configuration such that the inflatable member is only partially inflated. For example, in some embodiments, the inflatable member is inflated to a pressure below the diastolic blood pressure of the patient.

At 920, a sensor (such as sensor 220) is used to detect patient movement. For example, the sensor may detect if there is any patient movement that might cause or potentially contribute to an inaccurate blood pressure measurement. In some embodiments, the sensor may detect movement in a hand, arm, fingers, or other body part of the patient. If at 920 the sensor detects movement, a signal may be sent to controller. At 930, the inflatable member is prevented from further inflating. In some embodiments, the controller is configured to prevent the inflatable member from further inflation and to prevent a measurement of the blood pressure of the patient.

For example, the inflatable member may be prevented from inflating to the fully or more fully inflated state. In some embodiments, the controller (such as controller 230) is configured to receive the signal from the sensor that the sensor has detected movement of the patient. The controller may be configured to send a signal to a pump or a pump system to prevent the further inflating of the inflatable member and thereby prevent the measurement of the blood pressure of the patient at that time.

If the sensor does not detect patient movement, then at 940, the inflatable member may be allowed to be placed in the fully or more fully inflated state. For example, the controller may send a signal to the pump or pump assembly to place the inflatable member in a second or more fully inflated state. In some embodiments, the pressure within the inflatable member may be above a systolic blood pressure of a patient when the inflatable member is in the second or more fully inflated state.

Once the inflatable member is placed in the second or more fully inflated state, at 950, a blood pressure measurement of the patient may be taken once the inflatable member is placed in the fully or more fully inflated state. For example, in the illustrated embodiment, the sensor used to detect whether there was patient movement is used to measure the blood pressure of the patient. For example, in some embodiments, once the inflatable member is placed in the second or more fully inflated state, the inflatable member may be allowed to deflate. As the inflatable member deflates, the sensor may determine when vibrations associated with blood flow within the body of the patient are detected. According, a blood pressure of the patient may be determined. In some embodiments, the sensor communicates with the blood pressure measuring device to allow for a determination of the blood pressure of the patient. In some embodiments, the time to take or determine the blood pressure of the patient may be shorted as the inflatable member is already disposed in a partially inflated state before the blood pressure of the patient is measured.

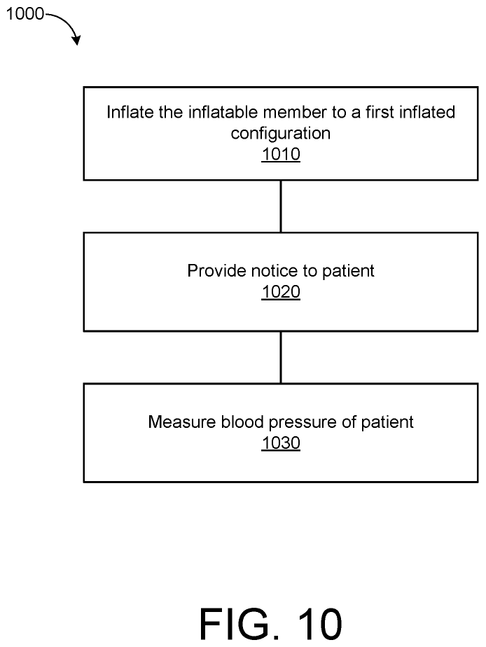

FIG. 10 is a flow chart of a method 1000 according to an aspect. At 1010, the inflatable member (such as inflatable member 210) is disposed proximate a portion of a body of a patient and is inflated to a first or partially inflated state. For example, the inflatable member may be inflated to a pressure below the diastolic blood pressure of the patient.

At 1020, the patient is notified that a blood pressure measurement is about to be taken. For example, in some embodiments, the partial inflation of the inflatable member generates a noise, such as an audible alert, and thus the patient is given an auditory notification that a blood pressure measurement is about to be taken. In some examples, the method 1000 can include providing notification to the patient that a blood pressure measurement is about to be taken with an alert provided by a display device, or haptic feedback. For example, a display device coupled to a device for obtaining, detecting, or otherwise receiving the blood pressure measurement can provide any suitable message, warning, or the like to the patient when a blood pressure measurement is about to be taken. In some examples, the device for obtaining, detecting, or otherwise receiving the blood pressure measurement can also provide any suitable haptic feedback, such as a vibration or a sequence of vibrations, to indicate to the patient that the blood pressure measurement is about to be taken. In some embodiments, the partial inflation of the inflatable member provides an amount of pressure on the body of the patient, thereby giving the patient a tactile notification that a blood pressure measurement is about to be taken.

In some embodiments, a patient may consciously refrain from moving when they know that a blood pressure measurement is about to be taken. This may allow for a more accurate measurement of the blood pressure of the patient. Additionally, when a patient has notification that a blood pressure measurement is about to be taken, the taking of the blood pressure measurement may not surprise the patient. According, it some cases, a more accurate blood pressure measurement may be taken.

At 1030, after the patient has been given notice that a blood pressure measurement is about to be taken, the blood pressure of the patient is measured. In some embodiments, the inflatable member is placed in a fully or more fully inflated state and allowed to deflate to measure the blood pressure of the patient.

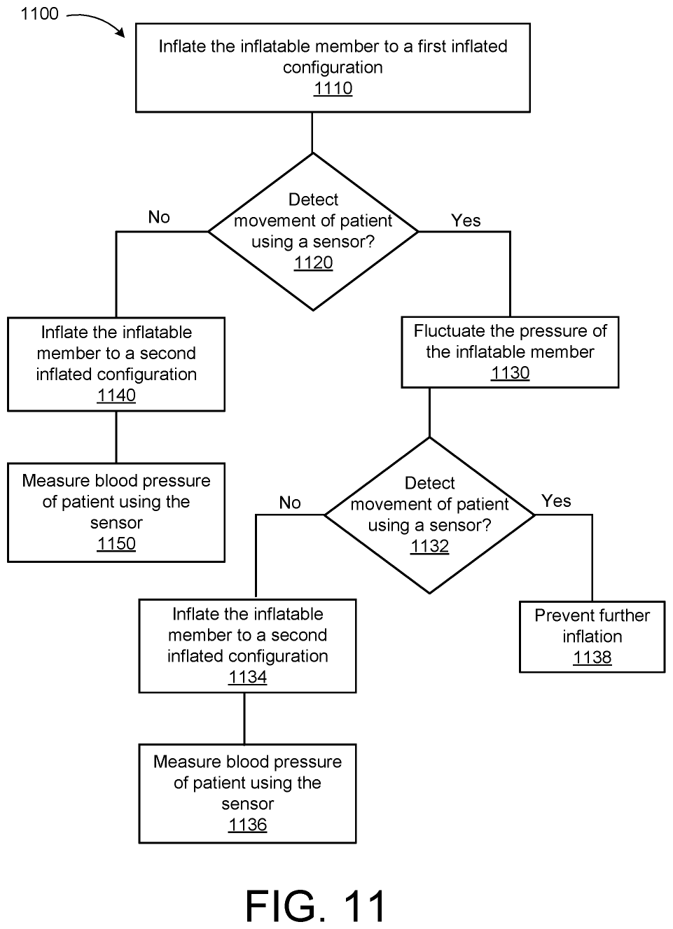

FIG. 11 is a flow chart of a method 1100 according to an aspect. At 1110, the inflatable member (such as inflatable member 210) is disposed proximate a portion of a body of a patient and the inflatable member is inflated to a first inflated configuration. In some embodiments, the inflatable member 210 is inflated to a first inflated configuration such that the inflatable member is only partially inflated. For example, in some embodiments, the inflatable member is inflated to a pressure below the diastolic blood pressure of the patient.

At 1120, a sensor (such as sensor 220) is used to detect patient movement. For example, the sensor may detect if there is any patient movement that might cause or potentially contribute to an inaccurate blood pressure measurement. In some embodiments, the sensor may detect movement in a hand, arm, fingers, or other body part of the patient.

If the sensor does not detect patient movement, then at 1140, the inflatable member may be allowed to be placed in the fully or more fully inflated state. For example, the controller may send a signal to the pump or pump assembly to place the inflatable member in a second or more fully inflated state. In some embodiments, the pressure within the inflatable member may be above a systolic blood pressure of a patient when the inflatable member is in the second or more fully inflated state.

Once the inflatable member is placed in the second or more fully inflated state, at 1150, a blood pressure measurement of the patient may be taken once the inflatable member is placed in the fully or more fully inflated state. For example, in the illustrated embodiment, the sensor used to detect whether there was patient movement is used to measure the blood pressure of the patient. For example, in some embodiments, once the inflatable member is placed in the second or more fully inflated state, the inflatable member may be allowed to deflate. As the inflatable member deflates, the sensor may determine when vibrations associated with blood flow within the body of the patient are detected. According, a blood pressure of the patient may be determined. In some embodiments, the sensor communicates with the blood pressure measuring device to allow for a determination of the blood pressure of the patient. In some embodiments, the time to take or determine the blood pressure of the patient may be shorted as the inflatable member is already disposed in a partially inflated state before the blood pressure of the patient is measured.

If at 1120 the sensor detects movement, a signal may be sent to the controller. At 1130, the controller causes the pressure within the inflatable member to fluctuate. For example, the controller may cause the pressure within the inflatable member in slightly increase and slightly decrease. The controller may cause the pressure within the inflatable member to slightly increase and slightly decrease several times over a short period of time. The fluctuation of the pressure within the inflatable member may provide notice to the patient that a blood pressure measurement is about to be taken. The notice to the patient may be tactile (feeling the inflatable member change pressure) or may be audible (hearing the inflatable member inflate and deflate). This notice may allow the patient to stop moving. For example, this notice may allow the patient to stop moving their hand or their arm.

At 1132, the sensor is again used to detect patient movement. For example, the sensor may detect if there is any patient movement that might cause or potentially contribute to an inaccurate blood pressure measurement. In some embodiments, the sensor may detect movement in a hand, arm, fingers, or other body part of the patient. If at 1132 the sensor detects movement, a signal may be sent to controller. At 1138, the inflatable member is prevented from further inflating. In some embodiments, the controller is configured to prevent the inflatable member from further inflation and to prevent a measurement of the blood pressure of the patient.

For example, the inflatable member may be prevented from inflating to the fully or more fully inflated state. In some embodiments, the controller is configured to receive the signal from the sensor that the sensor has detected movement of the patient. The controller may be configured to send a signal to a pump or a pump system to prevent the further inflating of the inflatable member and thereby prevent the measurement of the blood pressure of the patient at that time.

If the sensor does not detect patient movement, then at 1134, the inflatable member may be allowed to be placed in the fully or more fully inflated state. For example, the controller may send a signal to the pump or pump assembly to place the inflatable member in a second or more fully inflated state. In some embodiments, the pressure within the inflatable member may be above a systolic blood pressure of a patient when the inflatable member is in the second or more fully inflated state.

Once the inflatable member is placed in the second or more fully inflated state, at 1136, a blood pressure measurement of the patient may be taken once the inflatable member is placed in the fully or more fully inflated state. For example, in the illustrated embodiment, the sensor used to detect whether there was patient movement is used to measure the blood pressure of the patient. For example, in some embodiments, once the inflatable member is placed in the second or more fully inflated state, the inflatable member may be allowed to deflate. As the inflatable member deflates, the sensor may determine when vibrations associated with blood flow within the body of the patient are detected. According, a blood pressure of the patient may be determined. In some embodiments, the sensor communicates with the blood pressure measuring device to allow for a determination of the blood pressure of the patient. In some embodiments, the time to take or determine the blood pressure of the patient may be shorted as the inflatable member is already disposed in a partially inflated state before the blood pressure of the patient is measured.

Various implementations of the systems, such as the controller and other system, modules, and other units described herein, and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. Various implementations of the systems and techniques described here can be realized as and/or generally be referred to herein as a circuit, a module, a block, or a system that can combine software and hardware aspects. For example, a module may include the functions/acts/computer program instructions executing on a processor (e.g., a processor formed on a silicon substrate, a GaAs substrate, and the like) or some other programmable data processing apparatus.

Some of the above example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional step s not included in the figure. The processes may correspond to methods, functions, procedures, subroutines, subprograms, etc.

Methods discussed above, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a storage medium. A processor(s) may perform the necessary tasks.

Specific structural and functional details disclosed herein are merely representative for purposes of describing example embodiments. Example embodiments, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term and/or includes any and all combinations of one or more of the associated listed items.

The terminology used herein is for the purpose of describing particular embodiments only and is not intended to be limiting of example embodiments. As used herein, the singular forms a, an, and the are intended to include the plural forms as well, unless the context clearly indicates otherwise. It will be further understood that the terms comprises, comprising, includes and/or including, when used herein, specify the presence of stated features, integers, steps, operations, elements and/or components, but do not preclude the presence or addition of one or more other features, integers, steps, operations, elements, components and/or groups thereof.

It should also be noted that in some alternative implementations, the functions/acts noted may occur out of the order noted in the figures. For example, two figures shown in succession may in fact be executed concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the above example embodiments and corresponding detailed description are presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the above illustrative embodiments, reference to acts and symbolic representations of operations (e.g., in the form of flowcharts) that may be implemented as program modules or functional processes include routines, programs, objects, components, data structures, etc., that perform particular tasks or implement particular abstract data types and may be described and/or implemented using existing hardware at existing structural elements. Such existing hardware may include one or more Central Processing Units (CPUs), digital signal processors (DSPs), application-specific-integrated-circuits, field programmable gate arrays (FPGAs) computers or the like.

It should be borne in mind, however, that all of these and similar terms are to be associated with the appropriate physical quantities and are merely convenient labels applied to these quantities. Unless specifically stated otherwise, or as is apparent from the discussion, terms such as processing or computing or calculating or determining of displaying or the like, refer to the action and processes of a computer system, or similar electronic computing device, that manipulates and transforms data represented as physical, electronic quantities within the computer system's registers and memories into other data similarly represented as physical quantities within the computer system memories or registers or other such information storage, transmission or display devices.

Note also that the software implemented aspects of the example embodiments are typically encoded on some form of non-transitory program storage medium or implemented over some type of transmission medium. The program storage medium may be magnetic (e.g., a floppy disk or a hard drive) or optical (e.g., a compact disk read only memory, or CD ROM), and may be read only or random access. Similarly, the transmission medium may be twisted wire pairs, coaxial cable, optical fiber, or some other suitable transmission medium known to the art. The example embodiments not limited by these aspects of any given implementation.

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to employ the implementations in virtually any appropriately detailed structure. Further, the terms and phrases used herein are not intended to be limiting, but to provide an understandable description of the present disclosure.

It should also be noted that whilst the accompanying claims set out particular combinations of features described herein, the scope of the present disclosure is not limited to the particular combinations hereafter claimed, but instead extends to encompass any combination of features or embodiments herein disclosed irrespective of whether or not that particular combination has been specifically enumerated in the accompanying claims at this time. Additionally, while certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An apparatus, comprising:
an inflatable member configured to apply pressure to a portion of a body of a patient, the inflatable member being configured to be placed in a deflated configuration, a first inflated configuration, and a second inflated configuration, a pressure within the inflatable member when the inflatable member is in the second inflated configuration being greater than a pressure within the inflatable member when the inflatable member is in the first inflated configuration;
a sensor configured to detect vibrations; and
a controller operatively coupled to the sensor and to the inflatable member, the controller being configured to prevent the inflatable member from being placed in the second inflated configuration in response to the sensor detecting vibrations indicating movement of the body of the patient while the inflatable member is in the first inflated configuration.

2. The apparatus of claim 1, wherein the sensor is configured to detect vibrations associated with blood flow within the portion of the body of the patient.

3. The apparatus of claim 1, wherein the controller is configured to prevent the inflatable member from being placed in the second inflated configuration in response to the sensor detecting vibrations indicating movement of a hand of the patient while the inflatable member is in the first inflated configuration.

4. The apparatus of claim 1, wherein the inflatable member is a cuff and is configured to be removably coupled to an arm of the patient.

5. The apparatus of claim 1, wherein the inflatable member is configured to surround a portion of an arm of the patient.

6. The apparatus of claim 1, wherein the inflatable member includes an inflation portion and a coupling portion.

7. The apparatus of claim 1, wherein the controller is configured to receive a signal from the sensor when the sensor detects vibrations indicating movement of the body of the patient while the inflatable member is in the first inflated configuration.

8. The apparatus of claim 1, further comprising:
an output coupled to the controller and configured to provide a signal to inflate the inflatable member; and
an input coupled to the controller.

9. An apparatus, comprising:
a controller;
an output coupled to the controller and configured to provide a signal to inflate an inflatable member, the inflatable member being configured to apply pressure to a portion of a body of a patient, the inflatable member being configured to be placed in a deflated configuration, a first inflated configuration, and a second inflated configuration, a pressure within the inflatable member when the inflatable member is in the second inflated configuration being greater than a pressure within the inflatable member when the inflatable member is in the first inflated configuration; and
an input coupled to the controller,
the controller configured to prevent the inflatable member from being placed in the second inflated configuration in response to the input receiving a signal indicating movement of the body of the patient while the inflatable member is in the first inflated configuration.

10. The apparatus of claim 9, wherein the input is configured to be operatively coupled to a sensor configured to detect vibrations associated with blood flow within the portion of the body of the patient.

11. The apparatus of claim 9, wherein the inflatable member is a cuff and is configured to be removably coupled to an arm of the patient.

12. The apparatus of claim 9, wherein the inflatable member is configured to surround a portion of an arm of the patient.

13. A method, comprising:
inflating an inflatable member from a deflated configuration to a first inflated configuration to apply pressure to a portion of a body of a patient;
detecting movement of the body of the patient; and
preventing the inflatable member from inflating from the first inflated configuration to a second inflated configuration in response to detecting movement of the body of the patient.

14. The method of claim 13, wherein a pressure within the inflatable member when the inflatable member is in the second inflated configuration is greater than a pressure within the inflatable member when the inflatable member is in the first inflated configuration.

15. The method of claim 13, wherein the detecting movement of the body of the patient includes detecting moving using a vibration sensor.

16. The method of claim 13, wherein the detecting movement of the body of the patient includes detecting moving using a vibration sensor, the vibration sensor being configured to detect vibrations associated with blood flow within the body of the patient.

17. The method of claim 13, wherein the detecting movement of the body of the patient includes detecting movement of a hand or arm of the patient.

18. The method of claim 13, wherein the inflating the inflatable member provides notice to the patient that a blood pressure measurement may be taken.

19. The method of claim 13, further including:
coupling the inflatable member to an arm of the patient.

20. The method of claim 14, wherein a pressure within the inflatable member when the inflatable member is in the deflated configuration is less than a pressure within the inflatable member when the inflatable member is in the first inflated configuration.
